(19) United States
(12) Patent Application Publication
(10) Pub. No.: US 2016/0089384 A1
(43) Pub. Date: Mar. 31, 2016

(54) VITAMIN D-CONTAINING NUTRITIONAL SUPPLEMENTS

(71) Applicant: EVERBLOOM, LLC, Miami Beach, FL (US)

(72) Inventor: Leyda E. Bowes, Coral Gables, FL (US)

(21) Appl. No.: 14/868,685

(22) Filed: Sep. 29, 2015

Related U.S. Application Data
(60) Provisional application No. 62/056,667, filed on Sep. 29, 2014.

(51) Int. Cl. A61K 31/593; A23L 1/303; A61K 31/592

(52) U.S. Cl. CPC A61K 31/593; A61K 31/592; A23L 1/303; A23V 2002/00

(57) ABSTRACT

Provided are nutritional supplement preparations and methods for enhancing the outcome of a non-invasive tissue remodeling procedure, for providing the nutritional supplement to a subject, and for preparing such supplements.

### CROSS-REFERENCE TO RELATED APPLICATIONS

This application claims the benefit of U.S. Provisional Application No. 62/056,667, filed on Sep. 29, 2014, entitled “Vitamin D-Containing Nutritional Supplements,” which is hereby expressly incorporated by reference into the present application.

### FIELD OF THE INVENTION

The present application relates generally to nutritional supplements for supporting the body's response to non-invasive tissue remodeling procedures.

### BACKGROUND

“Subcutaneous tissue” includes tissue lying beneath the dermis and includes subcutaneous fat, or adipose tissue that may be composed primarily of lipid-rich cells, or adipocytes. Cellulite, or lumpy, bumpy, irregular “peau d'orange” or “cottage cheese”-like skin, forms when subcutaneous fat deposits into packets between bands of fibrous tissues that connect the dermis with the underlying tissues. Excess adipose tissue is thought to magnify the unattractive appearance of cellulite. The adipocyte has a large amount of cytoplasm that serves as a storage depot for excess triglycerides in the blood that are not utilized for energy. As adipocytes continue to enlarge within their intralobular and interlobular fascial compartments, they create “bulges” or convex distensions of soft tissue that then modify its contours.

Cellulite and excessive amounts of adipose tissue are often considered to be unappealing. Accordingly, a number of surgical and non-surgical procedures have been developed which remove such excess subcutaneous adipose tissue and cellulite.

Non-invasive body sculpting or tissue remodeling procedures have become popular, being safer, less traumatic and with quicker recovery, fewer side effects, and less discomfort relative to invasive or minimally invasive procedures such as liposuction, laser-assisted liposuction, and mesotherapy. Such non-invasive procedures include fat cooling methods (crylipolysis) (see, e.g., U.S. Pat. No. 7,367,341; U.S. Pub. No. 2005/0251120), suction and massage (U.S. Pub. No. 2008/0262574), and methods using radiofrequency energy (U.S. Pub. No. 2006/0036300), high frequency focused ultrasound (HIFU) radiation (see, e.g., U.S. Pat. Nos. 7,258,674 and 7,347,855), or low level light energy (U.S. Pat. No. 5,143,063) (see, e.g., Mulholland et al. (2011) Clin. Plastic Surg. 38:503-520). These procedures are thought to improve the look of fatty tissue by injuring adipocytes, the cells that store excess lipids. Damaged adipocytes may recover and heal or may eventually die if the procedure induces apoptosis, a process of programmed cell death, or if it negatively affects adipocyte size by decreasing lipogenesis (differentiation) or by stimulating lipolysis or adipocyte necrosis.

Despite being non-invasive, and hence causing less pain and a shorter recovery time, the results of these procedures take a longer time to fully develop. From one to six months is often the time quoted to see maximum improvement in the appearance of treated fatty tissue after the procedure. In addition, there have been side-effects noted, including paradoxical hyperplasia of the fatty tissue after cryolipolysis (Jalian et al. (2014) JAMA Dermatol. 150(3):317-9).

Thus, what are needed are methods and nutritional supplements which support and/or enhance the result of non-invasive tissue remodeling procedures while lessening the incidence of certain side effects.

### SUMMARY

It has been discovered that select concentrations of Vitamin D supports the body's natural responses to certain non-invasive injury. This discovery has been exploited to provide the present invention, which, in part, includes a non-invasive tissue remodeling procedure, thereby resulting in an improvement of the appearance of treated fatty tissue.

In one aspect, the disclosure provides a method of enhancing the outcome of a non-invasive tissue remodeling procedure, the outcome comprising an improved aesthetic appearance of a body area having undergone the procedure. This method comprises: examining the body area of the patient before the procedure; administering to the subject an amount of a nutritional supplement which supports the body's response to the procedure, and examining the body area after the procedure has been performed, the amount of nutritional supplement comprising Vitamin D at 400 IU to 10,000 IU wherein the treated body area has an improved aesthetic appearance relative to the appearance of the area before undergoing the procedure.

In some embodiments, the nutritional supplement administered comprises Vitamin D2, Vitamin D3, or a combination thereof.

In some embodiments, the step of examining the body area of a patient that has undergone the procedure is performed multiple times of increasing length after the procedure, and the improved aesthetic appearance of the treated body area is detected in a shorter period time after the procedure relative to the period of time it takes to detect an improved aesthetic appearance of a body area of a patient who has undergone the procedure and who has not been administered the nutritional supplement. In certain embodiments, the examining steps comprise photographing the body area and/or taking caliper measurements of the area. In some embodiments, the patient is weighed the patient before and after having undergone the treatment.

In certain embodiments, the nutritional supplement is administered before the procedure, and in particular embodiments, the supplement is administered daily from 1 day to 60 days before the procedure. In other embodiments, the nutritional supplement being administered after the procedure, and in particular embodiments, the supplement is administered daily from 1 month to 5 months after the procedure. In one embodiment, the nutritional supplement being administered before and after the procedure, and in other embodiments, the nutritional supplement preparation is administered on the day of the procedure. In particular embodiments, the nutritional supplement is administered before and on the day of the procedure, and in other embodiments, the nutritional supplement is administered on the day of the procedure, and after the procedure. In one particular procedure, the nutritional supplement is administered before, the day of, and after, the procedure. The supplement can be administered more than one time per day.

In some embodiments, the non-invasive tissue remodeling procedure performed is suction massage, suction massage and thermal energy, radiofrequency energy, high frequency-focused ultrasound energy, cryolipolysis, or low level light laser energy. In one embodiment, the non-invasive tissue remodeling procedure performed is cryolipolysis.

In another aspect, the disclosure provides a method of providing a nutritional supplement regimen to a subject, comprising administering to the subject one or more dosages of a nutritional supplement, the one or more dosages of the nutritional supplement providing: cholecalciferol at 400 IU to 10,000 IU.

In some embodiments, the dose of nutritional supplement is administered daily. In certain embodiments, the dose of nutritional supplement is delivered more than once a day. In particular embodiments, the dose of nutritional supplement is administered for from one day to eight months.

In yet another aspect, the disclosure provides a method of preparing the nutritional supplement preparations described above, comprising sieving each of the Vitamin D, genistein, resveratrol, and quercetin ingredients, mixing the component ingredients together, and then encapsulating the mixture in a dose form.

In still another aspect, the disclosure provides one or more dosages of a nutritional supplement for use in enhancing the outcome of a non-invasive tissue remodeling procedure, wherein one or more dosages of the nutritional supplement providing cholecalciferol at 400 IU to 10,000 IU. In some embodiments the dose of nutritional supplement is delivered more than once a day. In other embodiments, the dose of nutritional supplement is administered for from one day to eight months.

In another aspect the disclosure provides the use of one or more dosages of a nutritional supplement for enhancing the outcome of a non-invasive tissue remodeling procedure, wherein the one or more dosages of the nutritional supplement providing cholecalciferol at 400 IU to 10,000 IU.

### DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

Unless otherwise defined, all technical and scientific terms used herein have the same meaning as commonly understood by one of ordinary skill in the art to which this disclosure belongs.

It has been discovered that select concentrations of Vitamin D3 synergistically supports the body's natural responses to certain non-invasive tissue remodeling procedures. As such, this supplement is used as part of a method of enhancing a non-invasive tissue remodeling procedure.

### A. Nutritional Supplement Preparations

The nutritional supplement of the present disclosure has one active ingredient: Vitamin D. This compound has previously been shown individually to impact the adipogenic development of the mesenchymal stem cells, with simultaneous modulation of multiple molecular targets and effects on different stages of the adipocyte life cycle that ultimately translate in less adipose tissue.

In combination with Vitamin D, genistein can induce apoptosis and suppress lipid intake in maturing preadipocytes.

Vitamin D is a group of fat-soluble secosteroids responsible for enhancing intestinal absorption of calcium, iron, magnesium, phosphate, and zinc. Cholecalciferol (Vitamin D3) and its precursor, ergocalciferol (Vitamin D2) can be ingested from the diet and from supplements (Baile et al. (2011) Ann. N.Y. Acad. Sci. 1215:40-47; Kim et al. (2006) J. Nutr. 136:409-414; Aguirre et al. (2011) Open Nutraceuticals J4:189-198). The human body can also synthesize cholecalciferol in the skin from cholesterol with sun exposure. There are conflicting reports of the ability of Vitamin D to affect body fat mass and adipogenesis. For example, some studies have demonstrated that Vitamin D metabolites influence adipokine production and the inflammatory response in adipose tissue (Ding et al. (2012) Br. J. Nutr. 108(11):1915-23). Vitamin D has also been shown to have direct (non-calcemic) effects on adipocytes, and to suppress lipid accumulation and increase energy expenditure and fat oxidation. For example, Vitamin D supplementation to women during weight loss did not increase weight loss or associated factors compared with placebo (Mason et al. (2014) Am. J. Clin. Nutr. 99(5):1015-1025). In contrast, Vitamin D3 has been shown to stimulate adipogenesis in mice (Nimitphong et al. (2012) PLOS One 7(12):e52171.doi: 10.1371/journal.pone.0052171.Epub 2012 Dec. 18). Thus, the activity of Vitamin D appears to be variable, if not inconsistent.

However, Vitamin D has not been shown to support the body's natural processes in response to an invasive or non-invasive tissue remodeling procedure, thereby enhancing the aesthetic outcome of such a procedure, and resulting in an improvement in the visible appearance of treated tissues.

The present Vitamin D nutritional supplement appears to act synergistically to decrease the time it takes for certain initial and maximum results of the non-invasive tissue remodeling procedure to become visible, and to enhance the aesthetic appearance of the treated area. In the performance of a body sculpting procedure, certain fat cells and fatty tissues may undergo lethal or sub-lethal damage. The synergistic support of the present Vitamin D nutritional supplement works by both sensitizing the fat cells to the procedure damage and permitting the sub-lethal fat cell damage to become lethal, thereby improving procedure effectiveness. The Vitamin D nutritional supplement also enhances biological pathways in the body responsible for clearance of such released fat from the damaged adipose cells, leading to a more rapid observable procedure effect.

Vitamin D can be extracted from foods or can be commercially obtained. The supplement may contain other non-active ingredients as described below. All of these additional other ingredients are commercially available as well.

It is within the skilled artisan's purview to determine other optimal effective amount ranges for a subject undergoing a tissue remodeling procedure. The amount of Vitamin D which can be combined with a carrier material to produce a single dosage form will vary depending upon the subject undergoing the tissue remodeling procedure. The amount of Vitamin D which can be combined with carrier material(s) to produce a single dosage form, is generally that amount of the combined ingredients which produce a supportive effect.

For example, effective amounts of Vitamin D3 (cholecalciferol) are from about 400 IU to about 10,000 IU, from about 600 IU to about 5,000 IU, from about 1,000 IU to about 5,000 IU, or about 1,000 IU, about 2,000 IU, about 3,000 IU, about 4,000 IU or about 5,000 is provided as the serving and/or daily dose.

Vitamin D can be combined with a pharmaceutically-acceptable excipient, carrier, or diluent, depending on the form that the supplement takes. The phrase “pharmaceutically-acceptable excipient, carrier, or diluent” as used herein means a pharmaceutically-acceptable material, composition or vehicle, such as a liquid or solid filler, diluent, excipient, solvent, or encapsulating material, involved in carrying or transporting the main ingredients from one portion of the body to another part of the body. Each carrier must be “acceptable” in the sense of being compatible with the other ingredients of the formulation and not injurious to the subject. Some examples of materials which can serve as pharmaceutically-acceptable carriers include: sugars, such as food glaze, lactose, glucose, and sucrose; starches, such as maltodextrin, corn starch, rice starch, and potato starch; cellulose, and its derivatives, such as sodium carboxymethyl cellulose, ethyl cellulose, and cellulose acetate; powdered tragacanth; malt; gelatin; talc; excipients, such as silica dioxide cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring, and perfuming agents, preservatives and antioxidants can also be present in the compositions.

The nutritional supplement can be in a form suitable for oral administration. The formulations may conveniently be presented in unit dosage form and may be prepared by any methods well known in the art of pharmacy.

Nutritional supplements according to the disclosure suitable for oral administration may be in the form of capsules, cachets, pills, tablets, lozenges (using, e.g., a flavored basis, such as a sweetener), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of the four main ingredients of the present nutritional supplement.

In solid dosage forms for oral administration (capsules, tablets, pills, dragees, powders, granules, and the like), the four main ingredients are mixed with one or more pharmaceutically-acceptable carriers and/or any of the following: fillers, extenders, or excipients, such as starches, lactose, sucrose, glucose, mannitol, and/or silicic acid, and/or high molecular weight polyethylene glycols, and the like; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; coloring agents; and buffering agents.

Liquid dosage forms for oral administration of the compounds of the disclosure include pharmaceutically acceptable emulsions, microemulsions, solutions, suspensions, syrups, and elixirs. In addition to the four main ingredients, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof. Additionally, cyclodextrins, e.g., hydroxypropyl-beta-cyclodextrin, may be used to solubilize compounds.

Powders can contain, in addition to the four main ingredients, excipients, such as lactose, talc, silicic acid, aluminum hydroxide, calcium silicates, and polyamide powder, or mixtures of these substances.

In some cases, in order to prolong the effect of the nutritional supplement according to the disclosure, it is useful to slow the absorption of the main ingredients. This may be accomplished by using a thick or multiple pill coating(s) or by using very slowly dissolving capsule. The rate of absorption of the four main ingredients then depends upon the rate of dissolution of the coating or capsule.

Those with skill in the art are aware of methods of preparing pills, capsules, and the like. For example, to prepare the nutritional supplement in the form of a capsule, the initial manufacturing process entails dispensing or weighing the appropriate amounts of each of the ingredients, blending or mixing of the ingredients, and encapsulating the blended ingredients into empty capsule shells or forming it into pills, which are then coated, or into powders or crystals that can be dissolved or mixed in an aqueous solution.

### B. Non-Invasive Tissue Remodeling Procedures

The non-invasive tissue remodeling procedures which can be enhanced by the nutritional supplement according to the disclosure are many. These procedures tend to be classified on the basis of the type of energy delivered by a particular technology in modifying the adipocyte. Some of these procedures are shown below in Table 1 (Mulholland et al., supra).

TABLE 1

| Classification | Exemplary Procedure |
|---|---|
| Suction: Massage | Endermologie, France |
| Devices | |
| Suction-Massage: | TriActive (Cynosure, Inc., Westford, MA); |
| Thermal Devices | Smoothshapes (Cynosure, Inc., Westford, MA) |
| Radiofrequency | VelaSmooth, VelaShape (Syneron, Inc., Irvine, CA); |
| Energy Devices | Thermage# (Solta Medical, Hayward, CA); |
| | Accent (Alma Lasers Inc., Buffalo Grove, IL); |
| | TiteFX (Invasix, Inc., Yokneam, Israel) |
| High-Frequency | UltraShape (UltraShape Ltd., Yoqneam, Israel); |
| Focused | LipoSonix (Medicis, Scottsdale, AZ) |
| Ultrasound | |
| Energy Devices | |
| Cryolipolysis | Zeltiq (Zeltiq Aaesthetics, Pleasanton, CA) |
| (Cold) | |
| Energy Devices | |
| Low-Level Light | Zerona (Erchonia Medical, McKinney, TX) |
| Laser Therapy | |
| Devices | |

Most of these thermal technologies do not kill adipocyte or other surrounding cells in the fatty tissue. Some technologies deploy energy, either a pulse of high-voltage RF current, or a focused high-frequency ultrasound energy experience that disables or destroys the adipocyte by permanently damaging the cell membrane, or coagulating or disrupting and releasing the adipocyte cell contents such as low-level light laser therapy, create temporary disruptions in the cell membrane of the adipocyte allowing a temporary egress of the triglyceride from the cytoplasm, but the cell membrane then corrects itself. Accordingly, through the mechanisms of thermal augmentation of normal metabolic pathways, thermal destruction, cavitational destruction and/or an energy cascade and creation of a temporary adipocyte cell membrane pore, the final result is a temporary or permanent size reduction of the adipocytes and/or the number of adipocytes are reduced, which when translated over hundreds of thousands or millions of fat cells, can result in a measurable reduction of fat and a circumferential reduction of the body contour area in the treated area. The compositions according to the present disclosure can work to assist with any/all of these mechanisms, and thus with many different non-invasive body sculpting procedures involving thermal technologies.

### C. Cryolipolysis

One particular non-invasive tissue remodeling procedure that can be enhanced by the nutritional supplement according to the disclosure is cryolipolysis. A targeted area of a subject's body is drawn up with mild suction and the tissue is held between the panels of the treatment cup or the targeted area is pressed against a flat applicator consisting of cold panels for 30 to 120 minutes during which they apply cooling temperatures to the skin in a range of from about −20° C. to about 20° C. (see, e.g., U.S. Pub. No. 2010/0280582). The amount of cooling (selected energy extraction rate) is controlled by thermoelectric cooling cells powered by DC current and controlled by thermistors that monitor the skin temperature. Up to 20% of patients experience mild transient reduction in sensation (dysesthesia) that returned spontaneously with 7 weeks of treatment, but there are no reports of permanent sensory loss. (Mulholland et al., supra).

When cooling subcutaneous tissues to a temperature lower than 37° C., subcutaneous lipid-rich cells, such as adipocytes, can be selectively affected. In general, the epidermis and dermis lack lipid-rich cells compared to the underlying lipid-rich cells forming the adipose tissue. Because non-lipid-rich cells usually can withstand colder temperatures better than lipid-rich cells, the subcutaneous lipid-rich cells can be affected selectively without affecting the non-lipid-rich cells in the dermis, epidermis, and other surrounding tissue (see, e.g., U.S. Pub. No. 2010/0280582).

Without being bound by theory, the selective effect of cooling on lipid-rich cells is believed to result in, for example, membrane disruption, cell shrinkage, or disabling, destroying, removing, killing, or other method of altering lipid-rich cells. Cold exposure may also cause the onset of an inflammatory reaction within the treated adipose tissue. The mechanism for this phenomenon may be a cold-induced apoptotic adipocyte cell death for those fat cells that have been exposed to a cold stimulus that is above freezing but below body temperatures for a defined duration. Another mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte) (see, e.g., Mulholland et al., supra). Yet other mechanisms of injury may involve the lipid phase transition of those lipids within the cell's bi-lipid membrane, which results in membrane disruption, thereby compromising the integrity and/or function of the cell membrane and inducing apoptosis (see, e.g., Quinn (1985) Cryobiol. 22:128-147; Rubinsky (2003) Heart Failure Rev., 8:277-284). Local cold exposure also is believed to induce lipolysis (i.e., fat metabolism) of lipid-rich cells, and has been shown to enhance existing lipolysis which serves to further increase the reduction in subcutaneous lipid-rich cells (Vallerand et al., (1999) Aviation, Space Environ. Med. 70:42-50). Cold sensitivity of lipid-rich cells may be variable under certain circumstances and can lead to a certain variability of the threshold temperature needed to trigger the apoptotic or cell death event that is needed to lead to subcutaneous reduction of fat cells.

### D. Methodologies

An amount of the nutritional supplement preparation according to the disclosure is provided to the subject in one method according to the disclosure. For example, the preparation is provided for a certain period of time before undergoing a non-invasive tissue remodeling procedure. The subject also or alternatively takes that amount of the supplement for a certain period of time after the procedure. The preparation may also be provided on the day of the procedure.

In another method of the present disclosure, a supplement comprising Vitamin D3 (or Vitamin D3).

The amount of the nutritional supplement in the oral unit dosage form, with as a single or multiple dosage, is an amount that is effective for supporting a tissue remodeling procedure. The precise dose to be taken by the subject as directed by the physician performing the tissue remodeling procedure will depend on a variety of factors, examples of which include the specific type of tissue remodeling procedure being performed, as well as various physical factors related to the individual undergoing the procedure, such as their health and their level of bioavailable Vitamin D.

For example, the nutritional supplement preparation taken from one to about 60 days before the procedure, from 7 days to about 30 days before the procedure, from 5 to about 50 days before the procedure, from 4 to about 20 days before the procedure from one to about 14 days before the procedure, or 1 day, 2 days, 3 days, 4 days, 5 days, 6 days, 7 days, 8 days, 9 days, 10 days, 11 days, 12 days, 13 days, or 14 days before the procedure. The nutritional supplement preparation may also be taken from about 1 month to about 5 months after the procedure, from about 2 months to about 4 months after the procedure, from about 3 months to about 5 months after the procedure, or for about 1 month, about 2 months, about 3 months, about 4 months, or about 5 months after the procedure. The nutritional supplement preparation may also be taken on the day of the procedure.

In one method of the present disclosure, the body area of the patient who will be undergoing the tissue remodeling procedure is examined before and after the procedure. This body area is any part of the patient's body for which an improved aesthetic appearance is desired. For example, the targeted body area can be the hips, “love-handles,” abdomen, buttocks, arms, neck, flanks, and/or back. An improved aesthetic appearance of the targeted area may include a more consistent, even-textured, and smooth skin surface, and/or a decrease in the size of the targeted body area.

Examination can be performed by visual inspection of the body area which can be memorialized and further examined via photographic images. Measurements of the tissue in the body area can be taken, e.g., via calipers or other device known to those with skill in the art. The weight of the patient and other physiological measurements may also be taken. This examination can be performed multiple times of increasing length (e.g., days, weeks, months) after the procedure to measure the progress of the outcome of the procedure. It has been determined by such examination that administration of the nutritional supplement according to the disclosure results in an enhanced body response to the procedure (e.g., a shorter time to detect an improved aesthetic appearance of the area).

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Preparation of an Encapsulated Form of the Nutritional Supplement

The four component ingredients (Vitamin D3, genistein, trans-resveratrol, and quercetin dihydrate) as well as the other secondary ingredients listed were obtained from the commercial sources listed below.

| | | City, State |
|---|---|---|
| | | (or City, Country if |
| Ingredients | Manufacture | outside US) |
| Vitamin D3 850 CWS Powder | Supreem Pharmaceuticals Mysore Pvt. Ltd. | Geneva, |
| | | Switzerland |
| Silicon Dioxide (GL 200) | Glassven-Yangzhong Silicas and | Yangzhong City, |
| | Chemicals JV Ltd. | China |
| Dicalcium Phosphate | Jiangsu Chengxing Phosph-Chemicals Co. | Jiangsu, China |
| Dihydrate | Ltd. | |
| Magnesium Stearate | Faci Asia Pacific Pte. Ltd. | Singapore |
| Empty Hard Capsules of | CapsCanada | Pompano Beach, |
| Vegetable Origin Size “0,” | | FL |
| Natural | | |

Each of the component ingredients are tested for purity and released by Quality Control into the dispensing and blending area. The component ingredients are inspected (visually), weighed on calibrated scales (Precision scale-DWP-102 E) and placed in polyethylene bags for blending and mixing purposes. At this stage, each of the component materials are sieved (mesh size 10-40) to remove lumps or agglomerates. The components are then blended using a clean V-Blender (Patterson-Kelly cross flow blender 2 cubic ft) for at least 10 min. A lubricant (magnesium stearate) is then added and blending is carried out for a further 4 min. The blended material is collected in polyethylene bags and placed in drums and transferred to an encapsulation room. The equipment used for encapsulation is a semi-automatic CGN 208-D Capsule Filling Machine. The machine consists of two detachable pattern plates (one for the empty capsules shells and another for fitting caps onto the filled capsule bodies) and a hopper for filling the raw material blend. Once the capsule is filled with the blend, the two plates are brought together and through an in-built mechanism, the caps are fitted onto the bodies of the capsule. The finished capsules are then placed in the designated containers and desiccants may be added if required to these containers.

### Example 2

### Validation Study

A study was performed to demonstrate that a Vitamin D3 supplement enhances the efficacy of a non-invasive tissue remodeling procedure (e.g., cryolipolysis or “CoolSculpting™” (“CS”)) performed on a subject. Patients undergoing the procedure were instructed to take a Vitamin D3 supplement (e.g. Vitamin D3 Softgels, NOW® Foods, Bloomingdale, Ill.) daily for a period of 8 wks following the procedure.

The study consisted of 20 patients between the ages of 21 to 67 years old, with 15 patients being female patients and 5 being male patients. Each of the 20 patients had a minimum of 3 cycles of CS at 60 min per cycle (cycle duration) followed by 2 min of manual massage. In all areas treated there was an appropriate tissue draw. The common areas treated with CS in the 20 patients were: abdomen, flanks and back. Standardized baseline pictures were taken in all patients before the treatment as described above in Example 2. The patients were placed on Vitamin D3 supplementation for a period of 8 wks after the CS session, as follows: Vitamin D3 5,000 IU, 1 pill by mouth daily for 4 wks, then Vitamin D3 2,500 IU, 1 pill by mouth daily for 4 wks.

Comparing the group of patients receiving Vitamin D3 supplementation with all the patients treated before the study without Vitamin D3 supplementation, it was found that those receiving Vitamin D3 supplementation noticed results and reduction in the areas treated with CS as soon as 2 wks to 3 wks after the treatment, compared to those patients that do not receive supplementation and start noticed results 5 wks after the treatment. In addition, patients that received Vitamin D3 supplementation saw a 30% to 35% reduction in the areas treated with CS, compared to those who do not receive Vitamin D supplementation, and see an average reduction of between 20% to 25% of the fatty tissue. This represent a range of 20% to 75% enhanced results with supplementing with Vitamin D3, over those seen in patients without Vitamin D supplementation.

This, administering the Vitamin D3 supplement in the amounts and at the times described above resulted in an appreciable decrease in the time required to notice results of the CS treatment, as well as, a greater reduction of fatty tissue as compared to providing the CS treatment without supplementation.

### EQUIVALENTS

Those skilled in the art will recognize, or be able to ascertain, using no more than routine experimentation, numerous equivalents to the specific composition and procedures described herein. Such equivalents are considered to be within the scope of this disclosure, and are covered by the following claims.

1. A method of enhancing the outcome of a non-invasive tissue remodeling procedure, the outcome comprising an improved aesthetic appearance of a body area having undergone the procedure, the method comprising:
examining the body area of the patient before the procedure;
administering to the subject an amount of a nutritional supplement which supports the body's response to the procedure, the amount of nutritional supplement comprising Vitamin D at 400 IU to 10,000 IU; and
examining the body area after the procedure has been performed,
wherein the treated body area has an improved aesthetic appearance relative to the appearance of the area before undergoing the procedure.

2. The method of claim 1, wherein the step of examining the body area of a patient that has undergone the procedure is performed multiple times of increasing length after the procedure, and wherein the improved aesthetic appearance of the treated body area is detected in a shorter period time after the procedure relative to the period of time it takes to detect an improved aesthetic appearance of a body area of a patient who has undergone the procedure and who has not been administered the nutritional supplement.

3. The method of claim 2, wherein the examining steps comprise photographing the body area and/or taking caliper measurements of the area.

4. The method of claim 3, further comprising weighing the patient before and after having undergone the treatment.

5. The method of claim 1, wherein the nutritional supplement administered comprises Vitamin D2, Vitamin D3, or a combination thereof.

6. The method of claim 1, wherein the nutritional supplement is administered before the procedure.

7. The method of claim 6, wherein the supplement is administered daily from 1 day to 60 days before the procedure.

8. The method of claim 1, wherein the nutritional supplement being administered after the procedure.

9. The method of claim 8, wherein the supplement is administered daily from 1 month to 5 months after the procedure.

10. The method of claim 1, wherein the nutritional supplement being administered before and after the procedure.

11. The method of claim 1, wherein the nutritional supplement preparation is administered on the day of the procedure.

12. The method of claim 6, further comprising administering the nutritional supplement on the day of the procedure.

13. The method of claim 8, further comprising administering the nutritional supplement on the day of the procedure.

14. The method of claim 1, wherein the supplement is administered more than one time per day.

15. The method of claim 1, wherein the non-invasive tissue remodeling procedure is suction massage, suction massage and thermal energy, radiofrequency energy, high frequency-focused ultrasound energy, cryolipolysis, or low level light laser energy.

16. The method of claim 15, wherein the non-invasive tissue remodeling procedure is cryolipolysis.

17. A method of providing a nutritional supplement regimen to a subject, comprising administering to the subject one or more dosages of a nutritional supplement,
the one or more dosages of the nutritional supplement providing cholecalciferol at 400 IU to 10,000 IU.

18. The method of claim 17, wherein the dose of nutritional supplement is administered daily.

19. The method of claim 17, wherein the dose of nutritional supplement is delivered more than once a day.

20. The method of claim 17, wherein the dose of nutritional supplement is administered for from one day to eight months.
